(19) United States
(12) Patent Application Publication
(10) Pub. No.: US 2001/0014314 A1
(43) Pub. Date: Aug. 16, 2001

(54) Foot soap composition

(76) Inventor: Marie Harbeck, Austin, TX (US)

Correspondence Address: MARIE HARBECK, 3202 CLUMPGRASS COVE, AUSTIN, TX, 78735, US

(21) Appl. No.: 09/753,897

(22) Filed: Jan. 3, 2001

Related U.S. Application Data
(63) Continuation-in-part of application No. 09/248,573, filed on Feb. 11, 1999, now Pat. No. 6,193,987.

(51) Int. Cl. C11D 3/50; A61K 7/09; C11D 9/44; A61K 6/00; A61K 7/00; A61K 35/78; A01N 65/00

(52) U.S. Cl. 424/70.2; 510/101; 424/735; 424/736; 424/765; 424/768; 424/737; 424/776; 424/756

(57) ABSTRACT

An improved and useful organic oil-based foot cleansing composition and topical transdermal delivery system for hydrating and nourishing dry, cracked, itching, dermatitic, and eczemic skin infirmities, as well as the treatment and alleviation of fungal conditions of the foot, which has as its main constituents, safflower oil, flaxseed oil, boric acid, tincture of benzoin, and borax, in a glycerin base.

This is a “Continuation-In-Part” application for Ser. No. 09/248,573 filed Feb. 11, 1999 for Marie Harbeck.

### BACKGROUND

1. Field if Invention

This invention relates to an improved and useful organic oil-based foot cleansing composition as well as a topical transdermal delivery system for hydrating and nourishing dry, cracked, itching, dermatitic, and eczemic skin infirmities, as well as the treatment and alleviation of fungal conditions of the foot, which has as its main constituents, safflower oil, flaxseed oil, boric acid, tincture of benzoin, and borax, in a glycerin base.

To the best of the applicant's knowledge the following is the most relevant prior art:

U.S. Pat. No. 4,740,367 Force, et al. 1988

U.S. Pat. No. 5,244,679 Freston. 1993

U.S. Pat. No. 5,489,429 Griat, et al. 1996

U.S. Pat. No. 5,683,704 Ohba, et al. 1997

U.S. Pat. No. 4,883,659 Goodman, et al. 1989

U.S. Pat. No. 4,608,392 Jacquet et al. 1986

U.S. Pat. No. 5,431,911 Reynolds. 1995

U.S. Pat. No. 6,147,040 Van der Hagen. 2000

U.S. Pat. No. 6,143,704 Van Gunst et al. 2000

U.S. Pat. No. 6,046,145 Santora et al. 2000

U.S. Pat. No. 5,683,683 Scafidi 1997

U.S. Pat. No. 6,107,262 Nobles, II. 2000

The cosmetic and pharmaceutical industry is continually expanding its efforts in order to provide topical soap preparations that will cleanse, moisten, soften, eliminate dryness, cracking, chapping, chaffing, and redness of the skin, and to treat and prevent fungal conditions, and to restore the skin to its natural healthy condition.

Thus, synthetic skin aggravating surfactants, silicones, ammonia, solvents, acids, salts, artificial colors, and fragrances, and the like are being incorporated into the compositions of the cited prior art to extend shelf life of the cited compositions that are supplied to stores and supermarkets. Some of these components aggravate skin infirmities, thus the combinations of skin aggravating synthetic compositions employed continue to increase yearly.

It will be appreciated that the expansion and growth of the cited prior art has devolved into highly complex synthetic adjuvants for use in the formulations.

The problem with the use of the compositions of the cited prior art which make up the bulk of transdermal delivery systems are their chemical structures which are known to strip the acid mantle from the skin and contribute to foot irritations.

Alcohol, acids, solvents, synthetic perfumes, colorants, salts, and ammonia adjuvants incorporated into the compositions of the prior art cause dryness of the skin, rashes, allergies, dermatitis, peeling, bleeding, itching, painful cracking, dehydration and flaking of the skin.

Unfortunately, the cited prior art compositions are relatively ineffective for distressed skin conditions, not allowing the treatment and alleviation of skin irritations related to allergic skin reactions to synthetic adjuvants, so there is a need in the art for organic compounds for improved compositions for skin care.

The said prior art compositions do not employ the massive quantities of cold-pressed organic vegetable oils as in this invention.

The said prior art compositions do not employ potent organic compositions that this invention incorporates due to prohibitive manufacturing costs.

In terms of effectiveness, the said cited prior art compositions do not provide satisfactory skin penetration and supplying properties for treating fungal infirmities to leave skin satiny smooth and rehydrated.

The said cited prior art compositions do not provide the high volume of cold pressed organic safflower oil, or flaxseed oil, in their formulas to immediately restore polyunsaturated oil lipids that are essential to nourish and maintain healthy skin.

The said cited prior art compositions do not provide the high volume of skin healing vitamin A and tocopheral linoleate compositions to their compositions.

The said cited prior art compositions do not include tincture of benzoin as an antiseptic adjuvant in their compositions.

The said cited prior art compositions do not boil tincture of benzoin to eliminate its alcohol content.

The said cited prior art compositions do not provide an urgent need that exists for compositions like the present invention to the dermatological, pharmaceutical, alternative therapy and cosmetology fields.

The said cited prior art compositions do not provide organic compositions to alleviate and treat the dry skin symptoms of dermatitis, eczema, and lupus skin infirmities.

The said cited prior art compositions do not provide a mixture containing a high quantity of organic essential oil, borax, boric acid and tincture of benzoin to fight fungal infirmities of the foot.

The said cited prior art compositions do not provide boric acid to treat and prevent fungus related skin disorders.

The said cited prior art compositions do not provide organic polyunsaturated compounds that deposit oil lipids deep within the dermis while gently cleansing the feet during showering, washing or bathing.

The said cited prior art compositions do not provide organic moisturizing compositions that provide a protective water resistant, non-greasy, antiseptic and anti-fungal film on the skin.

The said cited prior art compositions do not provide organic compositions that are water resistant to body fluids, sebaceous gland activity and perspiration.

The said cited prior art compositions do not provide organic compositions to provide a high volume of organic essential oils as anti-itch, and coolant additives.

The said cited prior art compositions do not provide a liquid foot soap composition that can be used as a foot cleanser, anti fungal agent, and moisturizer.

The said cited prior art compositions do not provide a high volume of organic safflower and flaxseed oils in the said compositions.

The said cited prior art compositions do not provide a high volume of organic compositions to replace lost oil lipids to the dermis that are stripped by the solvent action of mineral oil based additives.

The said cited prior art compositions do not provide pleasantly scented essential oils to perfume their compositions.

Objectives and advantages of the present invention are:

a) to provide an alcohol free topical skin preparation,

b) to provide an essential oil as an organic antiseptic to treat bacterial, fungal and environmental aggressors of the skin;

c) to provide topical skin composition that has potent effectiveness in the treatment of skin infirmities, such as foot fungus, as well as chapping, rough skin, dryness, itching, cracking, flaking and skin allergies related to synthetic substances and the like;

d) to provide a high volume of organic cold pressed vegetable oil composition for immediate transdermal delivery,

e) to provide a satisfactory skin penetration of the said composition and supplying properties that leave the skin satiny smooth,

f) to provide a high volume of organic tincture of benzoin for anti-bacterial qualities and essential plant oil to perfume the said composition,

g) to provide a high volume of organic safflower and flaxseed oils in the said composition to replace lost oil lipids to the dermis,

h) to provide an urgent need that exists for compositions like the present invention to the dermatological, pharmaceutical, alternative therapy and cosmetology fields;

i) to alleviate and treat the symptoms of dermatitis, rough skin, dryness, cracking, eczema, chapping, itching and allergic skin reactions due to ammonia, solvents, acids, alcohol substances, and synthetic perfumes and colorants;

j) to provide a deeply moisturizing, and anti-fungal composition while gently cleansing the feet during bathing, washing or showering,

k) to provide an organic water resistant, non-greasy, antiseptic and moisturizing film on the skin,

l) to provide an organic composition that is water resistant to body fluids, sebaceous gland activity and perspiration;

m) to provide an organic composition without chemical structures that are known to cause skin irritations,

n) to provide an organic composition without solvents, mineral oil, artificial colorants and ammonia additives; and

o) to provide an organic composition suitable for the treatment and prevention of fungal infirmities of the feet.

An urgent need exists for skin compositions like the present invention, that employ a high volume of potent cold-pressed organic flaxseed and safflower oils plus organic vitamins to gently cleanse and feed the feet with nutritive polyunsaturated oil lipids without the addition of skin irritating synthetic adjuvants. Further advantages of this invention are the employment of organic essential plant oil, boric acid, borax and tincture of benzoin, that protect against fungal infirmities and fight environmental skin aggressors, leaving the skin in a healthy, hydrated, moist condition. Still further objects and advantages will become apparent from a consideration of the ensuing descriptions.

### BRIEF SUMMARY OF THE INVENTION

An improved and useful organic oil-based foot cleansing composition as well as a topical transdermal delivery system for hydrating and nourishing dry, cracked, itching, dermatitic, and eczemic skin infirmities, as well as the treatment and alleviation of fungal conditions of the foot, which has as its main constituents safflower oil, flaxseed oil, boric acid, tincture of benzoin, water, liquid menthol, tocopheral linoleate, oil of peppermint, sweet almond oil, apricot kernel oil, and borax, in a glycerin soap base.

### DESCRIPTION OF THE INVENTION

A liquid foot soap composition used to cleanse, treat and alleviate fungal conditions of the feet, and to restore the skin of the feet to a healthy condition through the transdermal delivery of organic nutrients and vitamins.

It is an objective of the improved invention to provide a potent, swift acting, organic foot cleansing formulation for treating and alleviating infirmities such as, cracking, itching, flaking and fungal skin conditions.

It is an objective of the improved invention to provide a liquid foot soap formulation that soothes, moisturizers and restores essential nutrients to the feet to improve the skins texture and protect the skin against dryness and cracking that is associated with fungal infirmities.

It is an objective of the improved invention to provide an improved organic foot cleansing formulation for the treatment and alleviation of itching and redness of the skin.

It is an objective of the improved invention to provide an improved liquid foot soap formulation for the treatment and alleviation of dry, chapped, cracked, flaky, itching and allergy prone skin.

It is still a further objective of the improved invention to provide an organic formulation for the treatment, alleviation and maintenance of callused, rough, skin whilst treating and alleviating fungal foot conditions.

These and other objects are accomplished in accordance with a particular embodiment of the present improved invention which provides a skin cleansing composition in the form of a liquid foot soap, comprising a mixture comprising tincture of benzoin, tocopheral linoleate, organic cold-pressed flaxseed, safflower, jojoba, sweet almond, and apricot kernel oils, glycerin soap, essential plant perfume, boric acid, and mixtures thereof The essential components of the present composition readily intermix to form a mixture suitable for topical cleansing, hydrating, and incorporates within its formula, potent anti-fungal properties. The present components are combined in such relative proportions so as to bring about a marked calming and anti-bacterial effect to the skin by supplying to the skin nutrients that are essential for healthy skin.

The present improved invention includes the surprising discovery that cold-pressed organic safflower and flaxseed oils, tincture of benzoin and high volume of tocopheral linoleate, can be used as active non-greasy principals in an improved foot cleansing composition to be employed in the treatment and alleviation of fungal foot conditions, while gently cleansing, and delivering organic vitamins, and linoleic oil lipids that are essential for the maintenance of healthy skin.

In accordance with the present improved invention there are provided formulations where cold-pressed, organic safflower oil, flaxseed oil, sweet almond oil, apricot kernel oil, tocopheral linoleate, essential oil of peppermint, and tincture of benzoin, can be employed to aid in the non-greasy treatment and alleviation of chronic dry skin, and fungal conditions. The embodiment of the improved invention follow to illustrate the formulation.

The main ingredients of my liquid foot soap are:

100 to 1000 grams glycerin soap base, purchased from S.F.J.C. Corp, 3241 Monument Way, Suite 1, Concord, Calif. 94518;

2 to 1000 milliliters water,

1 to 30 milliliters mysristamine oxide (Incromide Oxide M, purchased from Croda Inc),

0.1 to 100 grams boric acid granules,

2 to 200 milliliters cold-pressed safflower oil,

0.1 to 100 milliliters liquid menthol,

0.1 to 200 milliliters tocopheral linoleate,

1 to 80 milliliters essential oil of peppermint,

0.001 to 100 milliliter tincture of benzoin,

1 to 80 milliliters cold-pressed flaxseed oil,

1 to 80 milliliters sweet almond oil,

1 to 80 milliliters apricot kernel oil,

0.1 to 8 grams powdered borax,

0.5 to 2 milliliters dmdm hydantoin,

My liquid soap process is as follows:

placing glycerin soap base, flaxseed oil, saffloweroil, sweet almond oil, apricot kernel oil, tocopheral linoleate, and mysristamine oxide in a partially submerged container over hot water to form a mixture,

heating said mixture while stirring over hot water until dissolved,

simultaneously placing water, borax, boric acid, and tincture of benzoin, in another partially submerged container over hot water to form a mixture,

stirring said mixture over hot water until dissolved,

removing said mixtures from heat, and blending both mixtures by stirring until said mixtures cool to approximately 40 degrees C;

adding essential oil of peppermint, liquid menthol, and dmdm hydantoin to mixture, and stirring said mixture until it cools,

whereby the ingredients are formed into a liquid foot soap.

It is believed that the combination and high volume of organic cold-pressed flaxseed oil, safflower oil lipids, tocopheral linoleate, boric acid, borax, and tincture of benzoin, combined in the liquid foot soap, cleanse, restore and deliver nutrients that are essential for the maintenance of healthy feet, as well as alleviating, and treating conditions attributed to fungal foot infirmities, but we don't wish to be bound by this.

As noted, the improved composition hereof comprises a topical application which is applied to the dermal areas of the feet to alleviate and treat fungus, chapped, flaky, rough, reddened, cracked, and chronic dry skin conditions of the feet, by supplying to the skin materials with anti-fungal properties and the essential polyunsaturated oil lipids it needs to maintain its healthy condition.

The improved formulation is highly effective for cosmetic, alternative therapy, and pharmaceutical uses in the treatment of lupus, dermatitis, eczema and dry skin due to the resistance of the formulations to attack by body liquids, sebaceous oils and perspiration.

The improved formulation provides a long lasting moisturizing film on the skin that is applied while washing, showering or bathing the feet.

The improved formulation employs organic polyunsaturated oil lipids in the place of mineral oil in normal formulations, which by its solvent action strips skin surface lipids.

The improved formulation cleanses, hydrates, moisturizes the skin.

The improved formulation provides non-irritating anti-fungal properties to the skin.

The improved formulation does not strip the acid mantle from the skin.

The improved formulation is suitable for adolescent and aged care use.

The improved formulation treats skin in a pleasantly scented manner.

The improved formulation treats fungal foot infirmities while moisturizing and cooling the skin.

For a more complete understanding of the present improved composition reference is made to the following examples. The following examples are illustrative of the present improved composition and are not intended in any manner as a limitation upon the scope thereof

### EXAMPLE 1

The composition of the liquid foot soap was applied once daily to the feet of a man suffering from dry, cracked heels. User found the present invention did not sting the cracked areas of his heels as compared to commercially available liquid soaps.

### EXAMPLE 2

A female user, suffering from itchy, red skin between her toes, found the liquid foot soap calmed and cooled her skin, and within four days use, restored her skin to a healthy condition.

### EXAMPLE 3

To evidence the efficacy of the liquid foot soap composition, a series of individuals suffering from fungal foot conditions, were treated with the liquid foot soap formulation. Compared to commercially available liquid soaps, the results based upon the efficacy of the present invention are set forth hereinafter:

a) A female user found the present invention did not strip the natural oils from her skin and the effects lasted all day.

b) A female user found the present invention relieved the chronic fungal condition between the toes of her feet after daily application of present invention.

c) The present invention was used by a male sufferer, who found the skin on his feet did not dry out after daily use of the present invention.

d) All users found the present invention to have a deeply hydrating effect that remained on the skin until the next bathing period.

Thus, from the preceding it will be readily perceived by the reader that an urgent need exists for this improved composition for the alleviation of chronic skin conditions denoted heretofore, but which also, can be used to prevent such conditions from occurring;

The improved composition provides:

an organic, solvent free formulation,

an organic antiseptic to fight bacterial, and environmental skin aggressors,

a composition that has immediate effectiveness in the treatment of chronic dry skin infirmities,

a composition that has immediate effectiveness in the treatment of fungal foot conditions,

superior skin penetration and nutrient supplying properties,

high volumes of organic polyunsaturated oils that replace lost dermal lipids,

fills an urgent need in the pharmaceutical, alternative health, and cosmetic markets,

organic calming, moisturizing compounds that do not strip the acid mantle from skin,

deep moisturizing and cooling effects while cleansing the skin,

organic oil lipids in the place of mineral oil, which by its solvent action strips skin surface lipids; and

organic composition that leaves no drying residue on skin.

While the above description contains many specificity's, these should not be construed as limitations on the scope of the invention, but merely providing illustrations of some of the presently preferred embodiments of this invention.

Many other variations are possible. For example, it is additionally possible to add to the present invention many other adjuvants such as, color pearling pigments, additional plant oils and essential plant oil perfumes, oatmeal, almond meal, pumice, vitamins, antibiotics, anti-inflammatory additives, camphor, and lathering additives, floral waters, milk honey, vegetable, fruit and plant juices, dried vegetable and fruit peels, alpha-hydroxy, glycolic, lactic, salicylic, exfoliating fruit acids, and vegetable oils and be within the purview of the invention.

Accordingly, the scope of the invention should be determined not by the embodiments illustrated, but by the appended claims and their legal equivalents, rather than by the examples given.

What is claimed is:

1. A liquid foot soap comprising:
from 100 to 1000 grams liquid glycerin soap,
from 20 to 1000 milliliters water,
from 1 to 100 grams boric acid granules,
from 1 to 200 milliliters cold-pressed safflower oil,
from 1 to 100 milliliters sweet almond oil,
from 0.5 to 50 milliliters liquid menthol,
from 0.1 to 100 milliliters tocopheral linoleate,
from 1 to 80 milliliters of a perfuming compound chosen from a group such as oils of peppermint, spearmint, orange, lemon, lime, citrus, rose, lavender, bergamot, ginger, nutmeg, thyme, rosemary, neroli, woodrose, cedar, pine, grapefruit, peach, mulberry, raspberry and strawberry,
from 0.1 to 25 milliliters tincture of benzoin,
from 1 to 80 milliliters cold-pressed flaxseed oil,
from 1 to 80 milliliters apricot kernel oil,
from 0.03 to 6 grams powdered borax, and
from 0.5 to 2 milliliters of a preservative such as dmdm hydantoin.

2. A liquid foot soap comprising:
600 grams liquid glycerin soap,
30 grams boric acid granules,
15 milliliters cold-pressed safflower oil,
12.5 milliliters liquid menthol,
10 milliliters tocopheral linoleate,
10 milliliters of a perfuming compound chosed from a group such as oil of peppermint, spearmint, orange, lemon, lime, citrus, rose, lavender, bergamot, ginger, nutmeg, thyme, rosemary, neroli, woodrose, cedar, pine, grapefruit, peach, mulberry, raspberry and strawberry,
5 milliliter tincture of benzoin,
2.5 milliliters cold-pressed flaxseed oil,
2.5 milliliters sweet almond oil,
2.5 milliliters apricot kernel oil,
1.25 grams powdered borax, and 0.6 milliliters of a preservative such as dmdm hydantoin.

3. A liquid foot soap as in claim 2 further comprising approximately 5 to 80 milliliters of a foaming compound such as mysristamine oxide.

4. A method of making liquid foot soap comprising:
placing glycerin soap base, flaxseed oil, safflower, sweet almond oil, apricot kernel oil, and tocopheral linoleate, in a partially submerged container over hot water to form a mixture,
heating said mixture over hot water until dissolved,
simultaneously placing water, borax, boric acid, and tincture of benzoin, in another partially submerged container over hot water to form a mixture,
stirring said mixture over hot water until dissolved,
removing said mixtures from heat, and blend by stirring both mixtures until said mixtures cool to approximately 40 degrees C,
adding a perfuming compound such as essential oil of peppermint, liquid menthol, and a preservative such as dmdm hydantoin to said mixture,
and blending said mixture by stirring until it cools, whereby the ingredients are formed into a liquid foot soap.
